(19) United States
(12) Patent Application Publication
(10) Pub. No.: US 2010/0047332 A1
(43) Pub. Date: Feb. 25, 2010

(54) Treatments for burns using thyroid hormone compound in a human

(76) Inventor: Thomas Lavin, Watchung, NJ (US)

Correspondence Address: BROWN & MICHAELS, PC;400 M & T BANK BUILDING, 118 NORTH TIOGA ST, ITHACA, NY, 14850, US

(21) Appl. No.: 12/460,049

(22) Filed: Jul. 13, 2009

Related U.S. Application Data
(62) Division of application No. 11/455,971, filed on Jun. 20, 2006.
(60) Provisional application No. 60/693,813, filed on Jun. 24, 2005.

(51) Int. Cl. A61K 31/195; A61K 9/127; A61P 17/02

(52) U.S. Cl. 424/450; 514/567; 977/773

(57) ABSTRACT

A topical preparation for treating first and second degree burns includes TriAc. The topical preparation preferably includes less than 10 mg of TriAc per 100 ml of pharmaceutical excipient base. In another embodiment, a topical preparation includes TriAc for decreasing cellulite. In this embodiment, the topical preparation is preferably applied only before exercise. In a preferred embodiment, the preparation is applied only intermittently, preferably no more than three times a week, or in amounts sufficient to not downregulate receptor number and become inefficacious.

### REFERENCE TO RELATED APPLICATIONS

This is a divisional patent application of copending application Ser. No. 11/455,971, filed Jun. 20, 2006, entitled “TREATMENTS FOR BURNS AND ADIPOSE DEPOSITS USING THYROID HORMONE COMPOUND IN A HUMAN”, which claimed one or more inventions which were disclosed in Provisional Application No. 60/693,813, filed Jun. 24, 2005, entitled “TREATMENTS FOR BURNS AND CELLULITE USING THYROID HORMONE COMPOUND IN A HUMAN”. The benefit under 35 USC §119(e) of the United States provisional application is hereby claimed, and the aforementioned applications are hereby incorporated herein by reference.

### BACKGROUND OF THE INVENTION

1. Field of the Invention

The invention pertains to the field of topical thyroid hormone and thyroid hormone analogs, including burn treatments and subcutaneous fat and cellulite. More particularly, the invention pertains to thyroid hormone compounds applied topically at a non toxic concentration or amount in a discontinuous and intermittent fashion to reduce the amount of thyroid hormone exposure to the patient.

2. Description of Related Art

Thyroid hormones, thyroid hormone analogs and thyroid hormone like molecules interact with the human body via nuclear receptors and other physiological systems. In humans, this receptor C-erb-A protein family includes the human thyroid receptor alpha-1, the human thyroid receptor α2 which binds the hormone poorly or not at all, the human thyroid receptor β-1, and the human thyroid receptor β-2 and β-3 receptors. Receptors for thyroid hormones are found in all tissues within the human body, including human skin, heart and brain.

It has long been known that thyroid hormones also interact with the body via cell surface molecules, through altering mitochondrial chemical processes and via thyroid hormonal control of thyroxine metabolizing enzymes (the deiodinases), in addition to changing the metabolism of messenger RNA. Within any mechanism of action these hormones are considered to be iodothyronines which circulate within the human body and are composed of the thyronines: thyroxine T-4, tri-iodothyronine, Diodo-thyronione and the acetic acid deriviatives Tetrac, TriAc, and Diac. In addition to naturally occurring thyroid hormones, a large number of thyroid hormone acting agonists and antagonists (thyroid hormone like compounds or thyroid hormone like analogs) have been synthesized in the last 60 years and are the subject of numerous academic and patent publications. The presence of thyroid hormone itself in human skin has not yet been directly proven.

Many U.S. and international patents over the past fifty years describe a variety of molecular structures which have thyroid hormone like agonist or antagonist biological and nuclear receptor binding activity, usually described in rat anti goiter assays, an alteration of mitochondrial function, amphibian metamorphosis assays, or an increase in heart weight, a decrease in cholesterol or the ability to block hormone binding to its nuclear receptor and prevent a measured biological activity.

Some examples of thyroid hormone like structures include, but are not limited to, the structures disclosed in the following U.S. Pat. Nos. 6,979,750, 6,960,604, 6,803,480 6,794,406, 6,777,442, 6,608,049 6,555,582, 6,380,255, 6,326,398, 6,266,622, 6,236,946, 6,107,517, 5,883,294, 5,401,772, 5,061,798, 4,910,305, 4,826,876, 4,766,121. These patents are incorporated herein by reference.

Pharmaceutical effectiveness differs by compound and application. Differences in metabolism, half life, affinity at a particular pharmaceutical locus, toxicity to unrelated pharmaceutical targets, species differences, partition coefficients, molecular charge etc, all contribute to determining the usefulness of a particular compound.

Unfortunately, the effects of thyroid hormones in man are different from those in animals. For example, in amphibians, thyroid hormones affect regeneration and developmental changes, such as loss of a tail and metamorphosis. In rats, an excess of thyroid hormones increases growth hormone release from the pituitary, and growth hormone can modulate healing. No such increase in growth hormone is seen in humans. In chickens given exogenous thyroid hormones, growth hormone levels decrease, as shown in U.S. Pat. No. 5,168,102. In rodents, a large percentage of the total organism's thyroid hormone iodine metabolism occurs in the skin, while no such metabolism has been observed so far in humans. Rat models are not effective to determine thyroid hormone toxicity due to differences in thyroid hormone biochemistry as compared to humans and toxicologists urge investigators to use other models.

In addition to thyroid hormonal differences between animals and humans, basic genetic elements like corticotrophin releasing hormone receptor localization is vastly different between rodent and human skin, the architecture of the two portions of the skin, the dermis and epidermis are different as is the hair density and the permeability to many chemicals. Since stem cells which regrow the skin are present in the hair follicles, it would be logical to assume that rat and human models of wounding start from a very different baseline, with the rats having far more stem cells initially. It is also well known that humans heal differently from other mammals. In adult humans, a typical excisional wound may undergo a 20 to 30% contraction over a period of several weeks, while in other mammals the rate and extent of contraction is generally greater (for example up to 80-90 percent of wound closure is attributed to contraction in rats).

U.S. Pat. No. 3,198,702 discloses a method of treating burns to improve scar formation in rats, but no effect on healing time was observed. Both treated and untreated burns healed in 14 days. The patent discloses a topical burn-treating composition and method utilizing chemical compounds with healing properties, when applied locally to a burn in a rat. One of the compounds disclosed is triiodothyroacetic acid (TriAc).

All of the burns healed in 14 days, and the patent discloses no evidence that the treated burns healed faster. The postulated concentration range for effective compounds was between 0.01 and 5% and a preferred range of 0.1% to 3%. The patent specifically states that concentrations less than 10 mg/100 gms (0.01%) of cream have no effect, although the correct pharmacological predictor might be the amount of drug per square area (as recited in U.S. Pat. No. 6,221,911). The patent does not disclose how often the cream is applied or how much. This patent is hereby herein incorporated by reference.

Interruption of the stratum corneum of the epidermis leads to high absorption of drugs, by approximately 15 times. Since the amount of TriAc needed to make a 200 gm rat hyperthyroid is approximately 7 micrograms (Burger, European Journal of Endocrinology (1997) 137 537-544), application of even 100 μl of the formulations disclosed in U.S. Pat. No. 3,198,702 to the interrupted stratum corneum in the wounds would provide 200 micrograms of TriAc to the rat, 30 times the amount needed for hyperthyroidism. Subsequently, it has been shown that as little as 12 ug of T-3 or even less applied to the unbroken skin of a rat produces hyperthyroidism. Therefore, the concentrations used in this patent were more than enough to induce hyperthyroidism in the rats and produced systemic hyperthyroidism.

Given that the stratum cornea was broken, it is likely that a relatively large amount of drug would become systemic, resulting in a non topical effect. Subsequent research disclosed that adding small quantities of T-3 to the drinking water of rats is sufficient to make them hyperthyroid and to produce the same improved scarring effect as in U.S. Pat. No. 3,198,702, but no change in the healing rate in the burns in the rats subjected to excision and grafting after 5 weeks of time (Journal of Cutaneous Pathology Volume 1 Page 113-June 1974). No clinical demonstrations of utility for burns in humans for topically applied creams disclosed in this patent have ever been made, and no human pharmaceuticals for this purpose have ever been developed.

PCT published application WO 96/40048 and U.S. Pat. No. 6,221,911 show that thyroid hormone and thyroid hormone-like compounds, particularly TriAc, T-3, T-4 and other synthetic thyroid hormone analogues have substantially the same biological activity in human skin when applied in topical formulations. Both of these references are herein incorporated by reference. In established models of human skin, and human skin itself, gene expression arrays were used to compare gene expression produced by various doses of thyroid hormones and thyroid hormone like molecules and those of retinoic acid, glucocorticoids and vitamin D. TriAc was found to regulate expression of several genes that are important for skin structure and function. Other thyroid hormone compounds and thyroid hormone-like compounds also have similar effects as TriAc according to these tests.

U.S. Pat. No. 6,221,911 discloses that topically applied thyroid hormones or thyroid hormone like molecules produce a different phenotype in human skin than does the hyperthyroid state. Examples one through three disclose an induction of collagen genes and keratin 1 and TGF-beta with topical application to human skin or human skin substitute with a single application. Example 4 discloses treatment of excisional wounds in a rodent with a thyroid hormone. Other examples disclose epidermal growth effects of a topically applied thyroid hormone. The gene product alterations seen in U.S. Pat. No. 6,221,911 are associated with a more differentiated state, including collagen production.

U.S. Pat. No. 6,380,255 discloses a composition for topical application for dermal atrophy, dermal growth, pretreatment of dermatological wounds and for atrophy associated with diabetic dermopathy. An increase in activated fibroblasts is seen in treated human skin. The composition includes at least one thyroid hormone compound or thyroid hormone-like compound and a pharmacologically acceptable base. One of the thyroid hormone compounds disclosed in the patent is TriAc. This patent is hereby herein incorporated by reference.

Bukhonova et al., 1978 (Bukhonova, A. I. and Mirolyubova, Local Effect of Hormonal Factors on the Course of Repair Processes in the Skin of Irradiated Animals, Proceedings of a Conference of the OMSK Division of the All-Russian Scientific Society of Anatomy, Histology, and Embryology (1978)) discloses that the skin of irradiated animals may be healed using applications of hormones including deoxycorticosterone acetate and thyroxine applied to 1.5 cm by 1.5 cm excisional wounds (which contain dermis and epidermis only in the edges) in the animals. But, those skilled in the art know that irradiation typically makes animals hypothyroid. Bukhonova was merely supplementing them with the thyroid hormone that they were missing, and therefore, the animals healed better. Hypothyroid animals do not heal as well as normal animals. In contrast, hypothyroid humans have no trouble healing.

Such irradiated animals are functionally hypothyroid and any effects of thyroxine creams applied to excisional surface wounds merely replace thyroxine that is missing in the hypothyroid state. Safer et al (Thyroid, 2001, vol 11 p 717-724) have shown that topical application of topical T-3, but not intraperitoneally injected T-3, sufficient to change systemic T-4 levels, affect the growth of the dermis and epidermis in the mouse, as had already been known in the art.

Safer et al published that intraperitoneal T-3 improved wound healing in hypothyroid mice. (Endocrinology 145(5):2357-2361, 2004). T-3 also accelerates the healing of excisional non thermal wounds in the eurythroid mouse when used daily at an amount sufficient to modulate systemic T-4 levels in some of the mice. (Endocrinology 146(10); 4425-4430, October 2005).

On the other hand, Ladenson has published that hypothyroid humans have no defects in wound healing. (Ladenson P W, Levin A A, Ridgeway E C, Daniels G H 1984 Complications of surgery in hypothyroid patients. Am J Med 77:261-266).

U.S. Pat. No. 6,852,706 discloses a negative effect of thyroid hormones on the healing of rodent heart wounds, suggesting that thyroid hormone receptor activation and or cell surface molecule signaling via cell surface receptors leads to decreased collagen production and wound healing in rodents. Mice made hyperthyroid with 500 ngs of T-3 continuously applied in the form of a slow release pellet, which releases hormone over a period of at least several days, were seen to have decreased healing of heart wounds. Interestingly, thyroid hormone levels decrease acutely in humans after myocardial infarctions. Clearly the effect of thyroid hormone agonists on human skin displayed in this patent, is different from the effect of thyroid hormone on cardiac wounds in rodents.

The use of TriAc and its salts for a reduction of cellulite is disclosed in FR 2.153.202 (7134447). FR2197577 (72.30781) discloses various TriAc derivatives, including para hydroxy esters as having utility for the same purpose. EP060776 discloses activity of an isopropyl derivative of TriAc for reducing cellulite. CH642851 (1168/80) discloses a liposome formulation of TriAc together with glycosoaminoglycans for reducing cellulite. GB1354263 and BE784267 disclose the use of TriAc for reducing fat deposits. GB1400851 relates to the synthesis of ethyl esters and alkyl carboxy acids derivatives of TriAc and using them to reduce cellulite in combination with leeches, hyaluronidase, proteases, and lipase. FR2356427 discloses a treatment for cellulite applied every three days by ionizations, an undefined term, utilizing TriAc 100 mg/100 gm excipient together with enzymes and mucopolysacharides, but not TriAc alone. The term ionizations is not defined. FR2357246 discloses a composition for treatment for cellulite and sub dermal edema containing thyroxine at 20 mg/100 gm excipient requiring heparin, adrenaline, and enzymes and the remaining composition made entirely with a penetration enhancer in the composition.

U.S. Patent Publication No. 2004/0234592 discloses a composition including at least one thyroid hormone compound or thyroid hormone-like compound, a hydrophilic phase-forming component, an amino alcohol and at least two emulsifying or emollient excipients. TriAc is a preferred thyroid hormone or thyroid hormone-like compound in this application. This application is hereby herein incorporated by reference.

There is a need in the art for improved, safe and effective burn and dermal ulcer treatments, which decrease treatment and healing time. There is also a need in the art for topical treatments more effective, safer, and convenient in reducing subcutaneous fat deposits and/or cellulite, which do not cause hyperthyroid symptoms. This is especially important when thyroid hormones are applied to broken skin. The prior art does not deal with the concept of intermittent application of topical thyroid hormone and analogs.

### SUMMARY OF THE INVENTION

A topical preparation for treating burns includes TriAc. The burns being treated are preferably first and second degree burns. The topical preparation preferably includes a pharmaceutically effective amount delivering less than 10 mg of TriAc per 100 ml of pharmaceutical excipient base. In a preferred embodiment, the preparation is only applied intermittently.

In another embodiment, a topical preparation for decreasing subcutaneous fat includes TriAc. The topical preparation preferably includes between 1 and 20 mg of TriAc per 100 ml of pharmaceutical excipient base. The topical preparation delivers a pharmaceutically effective amount of TriAc, preferably less than 500 ng per cm². More preferably, the amount of TriAc is less than 200 ng per cm². In this embodiment, the topical preparation is preferably applied only before exercise. In a preferred embodiment, the preparation is applied only intermittently, preferably no more than three times a week.

In one embodiment, the topical preparation of the present invention preferably delivers less than 500 ng/cm² of TriAc. In other embodiments, adjustments are made for other compounds, vehicles and methods of delivery.

Another preferred embodiment provides an assay for the effectiveness for transdermal formulation of a thyroid cream.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 (Photo 1) shows a burn 4 days after the burn (Day 4), 24 hours after application of the TriAc topical preparation to a small area. The topical preparation had also been applied to the entire burned area 72 hours prior. The small area that had just been treated with the preparation 24 hours before can be seen as a lighter striped area having a reduction in redness within a darker background. A second application of drug to the entire area of the burn was performed just after this photograph was taken.

FIG. 2 (Photo 2) shows a picture of the burn five days after the burn (Day 5), 20 hours after application of the drug to the entire burned area on Day 4. The epidermis has turned brown and is beginning to slough. Even in the most deeply wounded areas, epithelialization has begun as denoted by the shiny appearance where the epidermis has been removed.

FIG. 3 (Photo 3) shows a photo taken six days after the burn (Day 6), approximately 48 hours after third application on Day 4. The entire wounded area has new regrown skin.

### DETAILED DESCRIPTION OF THE INVENTION

As little as 500 ug of thyroid hormone taken orally can induce sudden death in certain people. The present invention pertains to thyroid hormone compounds applied topically at a nontoxic concentration or amount in a discontinuous and intermittent fashion to reduce the amount of exposure. For the purposes of the present invention, intermittent is defined as less than once daily, and preferably less than three times per week.

For the purposes of this invention, a “thyroid hormone compound” or “thyroid hormone-like compound”, which terms are used interchangeably herein, is any chemical entity, including peptides, which binds to thyroid hormone receptor TRα or β with a dissociation constant, K_{d}, lower than 1 μM (which is the same as the inverse, an association constant, Kₐ greater than 10⁶) when tested in receptor binding assays, using pure or substantially pure natural or recombinant thyroid hormone α or β receptor containing the ligand binding domain or naturally occurring thyroid hormone receptor containing preparations such as solubilized rat nuclear thyroid hormone receptor. Such ligands may be considered agonists when they have similar agonistic effects as the natural hormone or may be considered antagonists when the compounds antagonize the effects of the natural hormone compounds. Partial agonist/antagonists also may exist. This provides a more readily standardized assay for classification purposes than for example an assay of amphibian metamorphosis, but does not assume any particular mechanism of action, nor any supposition that they are all acting pharmacologically in exactly the same manner, or have the same metabolism or the like. By using the above binding affinity stipulation, many compounds are excluded, as is known to the art.

TriAc is a thyroid hormone agonist with a potency about that of T-3, which is, additionally, slightly Beta selective in its binding. Although this application specifically discusses examples using TriAc, other agonist thyroid hormone compounds or thyroid hormone-like compounds could alternatively be used without deviating from the spirit of the invention since many have been shown to have similar skin effects and effective concentrations in biological assays. Some of these compounds, which can be tested in the above assay for appropriate binding affinity and tested for pharmaceutical effectiveness include, but are not limited to (using the nomenclature and biological actions found in Jorgensen, Thyroid hormones and Analogs, p 107-204, in Hormonal Peptides and Proteins, Ed. Cho Li, 1978, incorporated herein by reference): Tri-iodothyronine (3,5,3′-triiodothyronine, T3); D and L thyroxine (T4); 3,3′5′tri-iodothyronine (reverse T3); 3,3′-diiodothyronine; T3 and T4 analogues such as 3,5,3′,-Triiodo-L-thyronine methyl ester; 3,5,3′-Triodo-L-thyronine hydrochloride; L-thyroxine hydrochloride; Tetrac (3-[4-(4-hydroxy-3,5-diiodophenoxy)-3,5-diiodophenyl]acetic acid); Triac ([4-(4-hydroxy-3-iodophenoxy)-3,5-diiodophenyl]acetic acid); Tetraprop; Triprop ([4-(4-hydroxy-3-iodophenoxy)-3,5-diiodophenyl]propionic acid); T4Bu; T3Bu; Thyroxamine; Triiodothyronamine; (5-Benzyloxy-2-methoxyphenyl)-(2-methoxypyrimidin-5-yl)-methanol; Benzyloxy-2-methoxyphenyl)-(6-methylpyridin-3-yl)methanol; (5-Benzyloxy-2-methoxyphenyl)-(5-bromo-2-methoxypyridin-4-yl)methanol; (5-benzyloxy-2-methoxyphenyl)-(2,6-difluoropyridin-3-yl)methanol; (5-Benzyloxy-2-methoxyphenyl)-(2-methoxypyridin-4-yl)methanol; 4-Methoxy-3-[(2-methoxypyrimidin-5-yl)methyl]phenol; 4-Methoxy-3-[(6-methylpyrid-3-yl)methyl]phenol; 5-Benzyloxy-2-methoxybenzyl Bromide; (5-Benzyloxy-2-methoxyphenyl-(6-chloropyridazin-3-yl)-acetonitrile; 4-Benzyloxy-2-[2-methoxythiazol-5-yl)methyl]anisole; 6-[(5-Hydroxy-2-methoxyphenyl)methyl]thiazol-2-(3H); 3′-Heteroarylmethyl-4′-)-methyl-3,5-dinitro-N-trifluoro-acetyl-L-thyronine Ethyl Esters; 3′-heteroarylmethyl-3,5-di-iodo-4′)-methyl-N-trifluoro-acetyl-L-thyronine Ethyl Esters; 3′-heteroarylmethyl analogues of 3,3′,5-tri-iodo-L-thyronine (T3); 3′-substituted derivatives of the thyroid hormone 3,3′5-triiodo-L-thyronine (T3); L-3,3′-T2; DL-Br2I; L-Br2iPr; L-Me2I; L-Me3; L-Me4; L-Me2IPr; DL-IMeI; L-3,5-Dimethyl-3′-isopropylthyronine (DIMIT); DL-BPT4; B-triac; BP-tetrac; DL-SBT3; DL-SBT4; DL-MBT3; MB-tetrac; T2F; T2Cl; T2Br; T2Me; T2Et; T2iPr; T2nPr; T2sBu; T2tBu; T21Bu; T2Phe; T2F2; T2Cl2; T2Me2; 3,5,3′-Triiodo-D-thyronine; 3,5-Diiodo-4-hydroxyphenylpropionic acid (DIHPA); Aryloxamic acids; (arylamino)acetic acids; arylpropionic acids; arylthioacetic acids; (aryloxy)acetic acid; 3,3′-T2; 3,5-T2; 3′,5′-T2; alpha.-methyl-3,5,3′-triiodothyroacetic acid, alpha.-methyl-3,5,3′-triiodothyropropionic acid, and alpha.-methyl-3,5,3′,5′-tetraiodothyropropionic acid; methylene- and carbonyl-bridged analogs of iodinated thyronines or thyroacetic acids or iodinated benzofurans; 3,5-diiodo-4-(2-N,N-diethylaminoethoxy)phenyl-(2-butylbenzofur-3-yl)methan ol hydrochloride; [2-methyl-3-(3,5-diiodo-4-(2-N,N-diethylaminoethoxy)-benzoyl)benzofuran hydrochloride; [2-n-butyl-3-(3,5-diiodo-4-carboxymethoxy-benzoyl)benzofuran; 2-methyl-3-(3,5-diiodo-4-hydroxy-benzoyl)benzofuran; 2-methyl-3-(3,5-diiodo-4-carboxymethoxy-benzyl)benzofuran;] 4′-hydroxy-3′-iodo-3,5 diiodo-4-(2-N,N-dimethylamino-(ethoxy)benzophenone hydrochloride; 2-butyl-3-(3-iodo-4-hydroxybenzoyl)benzfuran; 4′,4-dihydroxy 3′3,5-triiodo-diphenylmethane; [3,5-d]iodo-4-(2-N,N-diethylaminoethoxy)phenyl-(2-butylbenzofur-3-yl)methanol hydrochloride; 2-methyl-3-(3,5-diiodo-4-(2-N,N-diethylaminoethoxy)-benzoyl)benzofuran hydrochloride; 2-n-butyl-3-(3,5-diiodo-4-carboxymethoxy-benzoyl)benzofuran; 2-methyl-3-(3,5-diiodo-4-hydroxy-benzoyl)benzofuran; 2-methyl-3-(3,5-diiodo-4-carboxymethoxy-benzyl)benzofuran; 4′-hydroxy-3′-iodo-3,5-diiodo-4-(2-N,N-dimethylamino-ethoxy)benzophenone hydrochloride; 2-butyl-3-(3-iodo-4-hydroxybenzoyl)benzofuran; 4′,4-dihydroxy-3′3,5-triiodo-diphenylmethane;] 3,5-diethyl, 3′-isopropyl thyronine (DIET); and IpTA2 (3,5 diiodo-3′isopropyl thyroacetic acid) and pharmacologically acceptable salts and derivatives thereof. In addition, compounds discussed in publication E. Morkin et al./Journal of Molecular and Cellular Cardiology 37 (2004) 1137-1146 and Borngraeber S, et al (2003) Ligand selectivity by seeking hydrophobicity in thyroid hormone receptor. Proc Natl Acad Sci USA 100:15358-153, analogs CGS 23425 and GC-1 and GC-24, or any other thyroid hormone-like compound may alternatively be used. Both of these references are hereby incorporated by reference.

The present invention preferably applies an amount of TriAc that is much less than a single oral replacement dose to a burn and significantly decreases healing time in humans without systemic toxicity. In a preferred embodiment, a dosage of approximately 1/20^{th} to 1/40^{th} of a normal daily replacement dose is used to treat a burn intermittently, resulting in a greater than 100% increase in healing rate.

The present invention discloses that small amounts of an endogenously occurring thyroid hormone, tri-iodothyroacetic acid (TriAc), applied twice to a burn approximately doubled the healing rate of a burn according to the time course given in the medical literature. The amount applied was less than 80 μg per 400 cm² or between 1/18th to 1/35th of an oral daily dose, which depending on the clinical setting varies from 20 to 40 ug/kilogram. In one embodiment, at least 48 hours are provided between applications of the hormone.

The present invention administers an amount of thyroid hormone administered topically far less than the human replacement dosage for the hormones on a daily basis, and vanishingly small on a weekly or monthly basis, by preferably applying the thyroid hormone intermittently.

The present invention also allows for the reduction of subcutaneous fat whether or not cellulite (defined herein as a dimpled appearance of the overlying skin) is present in the skin structures.

There are many prior art patents for cellulite, some discussing subcataneous fat or circumference changes. A 200 mg TriAc/100 g cream, to be applied twice a day, has been used in France for cellulite (which includes a skin permeability enhancer), but never used for burns or dermal ulcers. No one has reported reduced healing time for wound healing effects for TriAc, even though it has been on the market for 30 years. Although the same compound is used in France, Belgium, Argentina, Brazil for thirty years topically, there have been no disclosures to its medical efficacy or for advantageous subcutaneous fat reduction with exercise. Despite being known to the art for 40 years, there are no burn or dermal ulcer medications currently available using the thyroid hormone compounds or the thyroid hormone like compounds.

The TriaAc-containing creams of the present invention are produced by dissolving TriAc in a suitable solvent and adding it to a cream base. The cream preferably has a water content of 65 to 75%, with approximately 30% solids, and a pH of less than 7 (more preferably 5 to 6.3) by pH paper test sticks. Alternatively, other well known pharmaceutic carriers, either chemical or physical could also be used. For example, TriAc could also be applied to the oil phase of a water in oil or an oil in water cream base, applied as a patch (preferably a matrix patch), impregnated into a lattice or gauze, or charged in a solid lipid nanoparticle, or a liposome, as is known in the art. Other alternatives include physical delivery systems such as micro needle application or application accelerated by radio frequency microchanneling, which will generally require less thyroid hormone or thyroid hormone like compound.

The cream may be a semi solid emulsion such as an oil in water or water in oil cream as is known to the art, with anionic, non ionic, or cationic emulsification. An oil in water cream was utilized in the present examples. Since thyroid hormones and synthetic thyroid hormone like molecules may be carboxylic anions, phenolic anions, or zwitterions, or amines, or uncharged depending on the pH of the solvent and the chemistry of the thyroid hormone, the gallenic nature of the excipient will have to be adjusted, as is known to the art.

The compositions of the present invention may be conveniently supplied in a unit dose pack comprising a plastic container which may for example be bubble, or blow moulded or injected, together with a tear off plastic or foil top, the pack containing a unit dose of the composition. Some examples for amounts for the unit dose pack include, but are not limited to, 1 ml, 2 ml, 1 g, 2 g or 5 g.

In one embodiment, the compositions include less than 10 mg of TriAc per 100 ml of pharmaceutical excipient base. The compositions in this embodiment are preferably applied less than once daily.

In another embodiment, the compositions include less than 1 mg of TriAc per 100 ml of pharmaceutical excipient base or delivered with a physical delivery device. The compositions in this embodiment may be applied daily without resulting in a systemic response.

In one embodiment, the present invention includes TriAc in combination with beta adrenergic agonists, beta one selective or beta 2 selective or beta 3 selective such as dobutamine, dopexamine, albuterol or clenbuterol or Ractopamine or Salbutamol or ephedrine or Octopamine or SR 58611 (a preferential 3-adrenoceptor agonist), or even partial agonists such as CGP 12177, all contained within a suitable pharmaceutical base for topical application, preferably when used for a treatment of cellulite or the reduction of subcutaneous fat.

In another embodiment, a topical TriAc preparation includes TriAc; a pharmaceutical base suitable for topical application; and a compound selected the group consisting of yohimbine; forskolin and a combination of yohimbine and forskolin.

TriAc Burn and Deep Dermal Ulcer Treatment

A burn or deep dermal ulcer treatment including the thyroid hormone compound TriAc free acid decreases the treatment and healing time. The TriAc topical preparation is preferably used to treat burns which have not entirely destroyed the entire dermis. The TriAc topical preparation preferably contains less than 10 mg per 100 ml of pharmaceutical excipient base and are preferably applied at a concentration contacting the wound at a concentration of less than 160 micromolar. The preparation is preferably applied only intermittently, or discontinuously. In one embodiment, the cream is applied less than once daily. In a preferred embodiment, the TriAc preparation has a pH less than 7, and more preferably a pH of 5 to 6.3.

Commercially available pharmaceutical grade TriAc, which is a mixture of Diac, Tetrac and Triac and is generally composed of 0.2% to 0.5% Diac, 1.0 to 2% Tetrac, and 98.8 to 97.5 percent TriAc, was used in the examples herein.

The burn or deep dermal ulcer formulation, which is preferably in the form of a cream, may be improved with the use of hyaluronic acid and antibiotics such as bacitracin, neomycin, silver sulfadiazine and mafenide, or others if pharmaceutically compatible. Ions such as zinc and other factors such as vitamin can also be utilized.

The TriAc preparation of the present invention is preferably a semi solid cream or a liposomal preparation, for example in Novasome (IGI, Inc) or spray or solid lipid nanoparticle useful for the treatment of burns in humans. In one embodiment, the TriAc preparation is a water in oil or an oil in water emulsion of TriAc useful for the treatment of burns. In another embodiment, the TriAc preparation is a patch, or more preferably a matrix patch or is adsorbed onto a hydrocolloid dressing or other dressing.

In one embodiment, aloe vera is optionally added. In other embodiments, retinyl palmitate, panthenol and vitamin E, tocopherol acetate, d-alpha vitamin E and D, 1 alpha vit E, singly or in combination, are added to the topical preparation. For the purposes of this invention, Vitamin E describes any of a family of eight antioxidants: four tocopherols, alpha-, beta-, gamma- and delta-, and four tocotrienols (also alpha-beta-, gamma- and delta-) and their salts, bases and acids. These components preferably each have a concentration of less than 1 gm per 100 grams of delivery system.

The burn treatment of the present invention decreases the treatment and healing time dramatically. The healing time for a partial thickness second degree burn in the prior art is usually around 19 days (see Med Gen Med. 2001 Mar. 6; 3(2):3). No prior art discloses a healing time of 5 to 6 days, which is possible using the topical preparation of the present invention.

The dermal ulcer treatment of the present invention heals ulcers that were previously unhealing.

### Example 1

An initial experiment was performed with topical T-3 1 mg in 30 ml of Silvadene ointment. The cream was applied daily to a burned and blistered area of the right thigh for two weeks. Another area was treated with Silvadene ointment alone. The ointment was applied daily for two weeks in a very thin layer of less than 1 ml per application. Only a small change in epithelialization relative to the Silvadene ointment alone was seen, and production of more cellular debris and signs of inflammation occurred. After 6 weeks, the treated area showed less reddened areas and had less discolored skin. Other wounds such as lacerations, treated with either with topical T-3 or topical TriAc tended to show increased signs of inflammation with continuous application in approximately three days.

### Example 2

In this example, a young adult burn patient was treated with 7.5 mg of TriAc per 100 mg of cream base with a pH of 5.5 as measured by Ph PHast sticks (Merck). The water content was 65 to 75%, with approximately 30% solids. The semi solid cream formulation includes water, emulsifier, aloe vera, and less than 1% of d-tocopherol acetate or equivalent, panthenol, and retinyl palmitate.

On scalding burn day 0, ice was applied all night. The wound appeared as a large reddish area approximately 15 by 30 cm, with an area of white skin approximately 10 by 10 cm. The cream was applied to the entire area in an amount of much less than 1 gm (less than 80 μg per 400 cm²) on the evening of Day 1, 24 hours post burn. The worst blistering and numbness was over the whitish area. Over the next 48 hours, the wound became somewhat less painful and the white area started to become pink and then red. The outer area extending over the knee remained red. This burn was therefore a split thickness second degree burn with areas of third degree burn. There were no visible or other obvious indications that the first application had any effect.

The cream was applied again on the evening of Day 3, 72 hours post burn, only to a small area of most burned blistered skin over the original whitish area. The cream was applied in a striped fashion and produced a decrease in erythema and tenderness only in the area where it was applied. Prior to application of the cream, the entire area was brilliantly red.

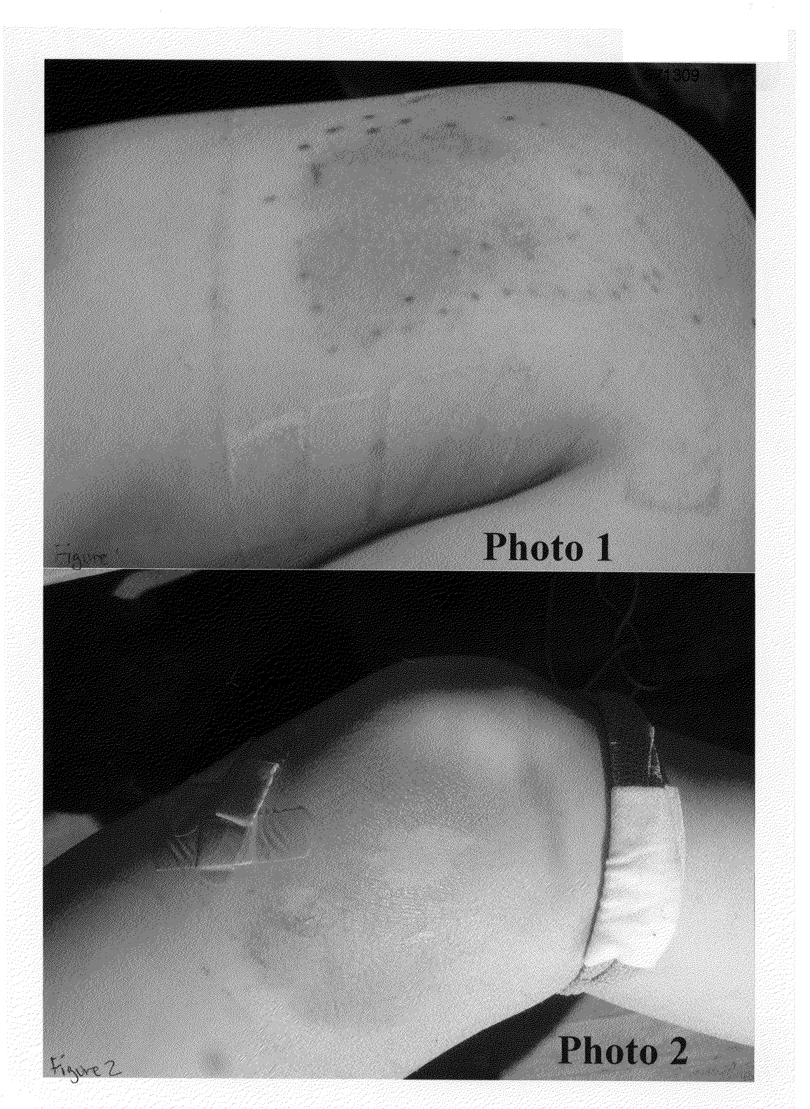

As shown in FIG. 1, this small application produced change in the wound in a pattern, which reflects the area in which it was applied, with less red skin within the small treated area, observed 24 hours after application. Blue marks were drawn outlining the wound. The inner set of marks shows the smaller more damaged area. Pain was also greatly diminished in this area, but continued unchanged in the other areas of the burn.

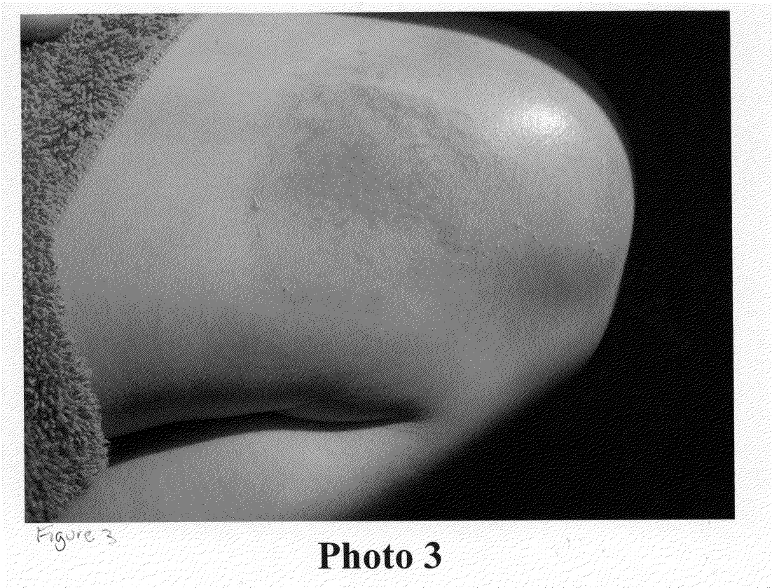

The cream was applied again on the evening of Day 4 over the entire area during this application. All pain was resolved overnight. FIG. 2 shows a photograph of the burn on day 5, 20 hours after this second application to the entire area. The entire area of the burn now had brownish epidermis in the process of being sloughed. The skin was virtually completely re-epithelialized. This was fourteen days less than the average prior art healing time of 19 days. There were no signs of inflammation or infection in the wound. FIGS. 1 and 2 are separated by approximately 36 hours.

There were no further cream applications. The next day, the remaining reddish brown epithelial layer sloughed.

FIG. 3 shows a photograph taken on Day 6, 48 hours after the last cream application on Day 4. This is six 24 hour periods from the time of the burn. Thus, in five to six 24 hour periods, a post scald second degree partial thickness burn, the burn completely re-epithelialized. The outline of the burn injury can clearly be seen by examining the margins of the wound.

The most affected area is noticeable six months after application as a small 4 cm by 5 cm more deeply pigmented area. Over a portion of this area, where the TriAc cream was applied less frequently, intermittent paresthesias continued for about 6 months. After 1.5 years, there is no pigmentation or scar.

Surprisingly, a cream with a concentration of 0.0075%, 25% lower than the 0.01% concentration taught in the prior art to be ineffective, applied discontinuously to the burn, in amounts not known to alter the TSH in an eurythroid human, even temporarily, when taken orally brought about rapid healing. In fact, pulsatile intermittent treatment healed the burn far more quickly than continuous T-3 treatment.

### Example 3

The patient was a 48 year old active triathlete who had developed a bicycle seat ulcer which was a persistent fissure running from coccyx almost to the anus. The deep dermal pressure ulcer resulted from pressure due to a bicycle seat. This pressure injury frequently was oozing and was generally moist. The fissure was 1-2 mm wide and 2-3 mm deep. It was resistant to a variety of over the counter steroid and anti-fungal creams and lanolin. Treatment with Oxystat for a yeast infection also failed to resolve the matter. While sometimes partially healing, it persisted for about 2 months before treatment with TriAC 0.006% (6 mg/100 gm) cream. The cream was applied for 4 treatments on an every other day basis, using barely enough to treat the area about 20 cm², sometimes every third day. Treatment resulted in substantial healing over the next few weeks. Skin has remained intact for close to 6 months. The dermal ulcer in this example was non healing until the cream was used on an intermittent basis

Treatment of Cellulite and Subcutaneous Fat Reduction.

Topical preparations, such as creams, including TriAc reduce cellulite and subcutaneous fat deposits in humans when applied prior to exercise and in an intermittent, discontinuous fashion. In a preferred embodiment, the discontinuous application is application less than once daily. In a more preferred embodiment, the discontinuous application is application three or less times per week. In a preferred embodiment, exercise is performed between 30 minutes and 2 hours after application of the preparation.

In another preferred embodiment, the exercise is performed between 30 minutes and 1 hour after application of the preparation.

In another preferred embodiment, the TriAc preparation has a pH less than 7, and more preferably a pH of 5 to 6.3. In a more preferred embodiment, the total number of applications required to produce a visible result in reduction of cellulite or decreased subcutaneous fat is fewer than 30 applications, and often fewer than 20 applications.

A package label or directions for a TriAc preparation states apply only before exercise.

In one embodiment of the present invention, a glycerol assay is used to determine the effectiveness of topically applied thyroid hormones or thyroid hormone like molecules as described in the art to both cross the dermal epidermal barrier and to modulate lipolysis of fat when topically applied. More specifically, this embodiment preferably uses a glycerol assay to determine the effectiveness of topically applied thyroid hormones or thyroid hormone like molecules as described in the art to modulate lipolysis of fat when topically applied in an amount less than a single oral dose used typically for daily therapeutic or replacement dosages. An enzymatic or luminescent plasma glycerol assay, or a glycerol assay determined by other methods known to the art, such as GC-MS (Bernert et al, J. Chromatography, 578, p 1-7, 1992, incorporated herein by reference) is preferably used to determine the effects of thyroid hormones.

The present invention shows that topical TriAc has strong effects on lipolysis. Prior application of topical TriAc significantly alters the human plasma glycerol level after exercise in the fed state and thus topically applied TriAc and thyroid hormone like agonist analogs, can induce a significant degree of lipolysis in humans. Surprisingly, the present invention shows that application of 120 ug of TriAc topically to greater than 500 cm² of skin is capable of dramatically altering the concentration of circulating glycerol (a measure of fat breakdown) when applied prior to low intensity exercise. The amount of thyroid hormone or thyroid hormone like agonist applied to the skin is approximately 1/10th the amount of a total single daily oral dose taken as a replacement or therapeutic dosage. The ability of topical TriAc to reach and affect the adipose tissue as disclosed herein may be the result of the effect of exercise on human skin, or the metabolic endocrine alterations which accompanies exercise.

In the prior art, either pictorial, patient rating scales, or measurements of limb circumference have been used to indirectly measure the effectiveness of topical TriAc for treatment of cellulite and adiposity. These measures do not prove an effect directly on the fat tissue.

The standard assay for thyroid hormonal effects in humans is the TSH test (Advisory Committee for Pharmaceutical Science Briefing Document Levothyroxine Bioequivalence Advisory Committee Meeting—12-13 Mar. 2003; FDA. Transcript of the Meeting of the Advisory Committee for Pharmaceutical Science, Mar. 13, 2003, Food and Drug Administration Public Meeting on Levothyroxine Sodium Therapeutic Equivalence May 23, 2005, incorporated herein by reference). TSH is the most sensitive and widely available measure of small perturbations in serum thyroid hormone levels. A two fold change in T-4 levels provides a ten fold change in TSH.

Glycerol assays are useful in determining the lipolysis due to pharmacologic agents when these pharmacologic agents are applied directly to fat tissue or when given orally or parenterally, and have been available for at least 25 years for use with T-3 and T-4 and other hormones and during exercise. For example Pfeifle et al, Hormone Metab. Res. 12, p 711, 1980 used glycerol assays to determine effects of thyroid hormone during adrenergic stimulation of adipocytes in cell culture. Hellstrom et al Journal of Clinical Endocrinology and Metabolism Vol. 82, No. 1 1997 investigated the effects of additional adrenaline on isolated adipocytes in cell culture from hyperthyroid people. Henrik et al, The Journal of Clinical Endocrinology & Metabolism Vol. 87, No. 11 4966-4975 2002 investigated the effects of acute growth hormone administration on plasma glycerol in trained athletes during exercise in the fasted state. No assay of plasma glycerol after transdermal delivery of drug directly applied over subcutaneous fat tissue has been obtained. These references are all incorporated by reference.

The application of the glycerol assay to a topical or transdermal application of a therapeutic agent has never been achieved prior to the present invention, because it has been believed that no topical agent could affect lipolysis without either affecting systemic metabolism or simply being pharmaceutically ineffective. The present invention shows that application of 120 ug of TriAc topically to greater than 500 cm² of skin is capable of dramatically altering the concentration of circulating glycerol when applied prior to low intensity exercise.

This is below the minimum amount of drug ever previously given to a human. TSH is exquisitely sensitive to Triac administration in humans. The minimum oral dosage of this drug which affects human physiology by transiently changing TSH levels by only 30% over a few hour time period is 350 ug (Menegay, Juge and Burger, ACTA Endocrinologica, 1989, 121 p 651-658). No human effects of this drug at a dosage level of 120 ug using any sort of application, parenteraly or oral or topical, have ever been seen before. No permeation enhancers of any sort have been used in the topical preparation. Furthermore, it has recently been shown that no effect on TSH levels, or other thyroid hormone function tests such as serum T-3 and T-4, occur in humans from topically applied thyroid hormones, either T-4 or TriAc, (Santini et al The Journal of Clinical Endocrinology & Metabolism 88(6):2825-2830, 2003, Yazdanparast et al, THYROID, 14, 5, p 345 2004) despite application of many fold higher amounts of thyroid hormone and in the case of TriAc, using similar formulations, but much higher applied dosages. Thus plasma glycerol levels are an assay of thyroid hormone function independent of TSH.

### Example 1

TriAc-containing creams were produced by dissolving TriAc in a suitable solvent and adding it to a cream base. The cream had a water content of 65 to 75%, with approximately 30% solids, a 5.5 by pH paper test sticks, color pHast (Merck). TriAc could also be added to the oil phase of a water in oil or a oil in water cream base, or charged in a solid lipid nanoparticle, or applied as a liposome, or a matrix patch as is known in the art.

In this example, a 27 year old non obese female applied creams containing between 8 mg and 16 mg of TriAc free acid per 100 ml of excipient, to the right upper posterior thigh and right buttocks. The cream was only applied before exercise. More specifically, application occurred between 30 minutes and 2 hours before mild exercise. The mild exercise involved the underlying musculature, and included long walks, jogging, or gym exercises involving light resistance exercises, or stairmasters, treadmills and the like.

The amount of cream applied was always less than 5 ml, just sufficient to cover the area, usually less than 2 ml. Thus, less than 0.16 mg to 0.3 mg of TriAc was applied per application. For most of the applications, the amount of cream applied was less than 3 ml and usually less than 1.5 ml. In these applications, less than 0.4 mg was applied and as little as 80 μg was applied in at least some of the applications. Exercise occurred one to three times a week. Consequently, the cream was only applied one to three times a week. Two grams of cream, approximately 2 mls, was sufficient to cover the buttocks, back of thighs, and anterior waist area.

A white light 3-d whole body scanner, such as made by TC2, or a scanning laser 3d body scanner such as that made by Human Solutions, or a photographic 3-d imaging processing system such as made by Inspeck or Visimage systems can be used to produce time serial measurements of human body circumferences and volumes to assess the utility of products which alter human body contours and shapes and cellulite and subcutaneous fat in a clinical study.

After twelve applications, a noticeable change was observed. The skin was smooth and little evidence of cellulite remained where it had been previously present. The contour of the buttocks was smaller and smoother and clothing fit more loosely. Such a result is obtainable with fewer than 30 applications and less than 100 ml of applied cream.

For the practice of this invention, it is preferred that applications occur no greater than three times a week.

### Example 2

A 40 year old non obese female who ran 20 miles a week had lateral hip fat deposits which were resistant to any change. 1.5 to 2 ml of cream, containing 9 mg TriAc per 100 ml base, was applied to these areas about 30 minutes before exercise between 1 and 3 times a week followed by exercise, usually running. Little change was observed for 2 to 3 weeks. However after 5 weeks of application, the fat deposits were noticeably different such that they could barely be discerned.

### Example 3

Compared to the formulation herein, commercial products have between 16 to 33 times the concentration and are applied twice a day instead of two to three times a week. The present invention shows that a 70 to 140 fold decrease in the amount of thyroid hormone applied per week produces substantial results with an increase in safety over the prior art. This example shows some biological data supporting the previous examples.

Glycerol is formed in the blood from the breakdown of triglycerides, a kind of fat stored inside fat cells. Glycerol concentrations increase in the bloodstream of trained atheletes during exercise, especially after an overnight fast. It is a measure of the breakdown of fat cells.

This example uses a glycerol assay to determine the effectiveness of topically applied thyroid hormones or thyroid hormone like molecules as described in the art to increase lipolysis of fat when topically applied. More specifically, this example uses a glycerol assay to determine the effectiveness of topically applied thyroid hormones or thyroid hormone like molecules as described in the art to dissolve fat when topically applied in an amount less than a single oral dose used typically for daily therapeutic or replacement dosages.

Three adult humans (A, B, and C) with normally functioning thyroids and no history of cardiovascular disease, varying by age, sex, BMI, body surface area, and physical activity, were tested. Age varies from less than 25 (subject A) to over 40 (subjects B and C). BMI varied between 18 (A), 20 (C), and 30 (B). Physical activity was greatest in subject A, who had the lowest BMI and was also the youngest. The other two subjects typically had less than three hours of exercise per week. No food or drink other than water was given two hours prior to application of the TriAc formulation. With this short fast, the subjects are still in the fed state and plasma glycerol levels would be expected to not change during exercise, as taught in the literature for young fit subjects (Coyle et al, J Appl Physiol. 1985 August; 59(2):429-33, herein incorporated by reference). The fed state as defined herein is an individual not having an overnight fast. Topical TriAc was applied as in Examples 1 and 2, by applying a weighed amount of semisolid cream formulated to contain 120 micrograms of TriAc to the right and left posterior thighs, buttocks and anterior abdominal areas, a surface area much greater than 600 sq cm. The amount of cream and thus TriAc amount was not varied according to age, sex, or weight. The cream was applied intermittently: application days were separated by at least two days. Dosages other than in this example, e.g. amounts of TriAc per cm² either above or below this level may alternatively be utilized, especially with changes in the pharmaceutic vehicle or delivery device.

A venous plasma glycerol sample was obtained 35 to 40 minutes prior to mild exercise, and either the TriAc containing cream or a non TriAc containing identical cream was applied. The subjects rested for 30 minutes and then exercised in a defined manner to maintain either 65-70% of their age adjusted maximal heart rate or 110-120 strides on a Precor 544 EFX elliptical trainer for 30 minutes. Heart rate was monitored continuously with a Polar wireless monitoring device. Room temperature varied between 72 and 84 degrees. Calories expended (from Precor) per kilogram were approximately constant among each participant and between participants on a per kilogram basis during each of the exercise sessions and were usually around 3.0 to 3.5 calories per kg (see Table 4). After a 30 minute exercise session and a 5 minute programmed cool down session, another plasma glycerol assay was taken. The samples were immediately refrigerated and centrifuged after 0 to 10 hours, each exercise set treated exactly the same way, frozen at −40° C. and assayed with the Sigma-Aldrich plasma glycerol assay after deproteinization. Exercise time was fixed at a regular time of day. The next day, the experiment was repeated. The order was placebo day, then treatment day.

The magnitude of the change pre and post exercise with and without TriAc was calculated and the percentage change from baseline with and without TriAc was calculated. Topical TriAc produced large effects. The youngest participant had the most stunning changes, suggesting that fat tissue becomes less sensitive to thyroid hormone induced lipolysis as people age, possibly due to the effects of growth hormone or other hormones or cytokines, or that response increases with cardiac output. Remarkably, Triac produced a change in the exercise induced plasma glycerol levels during this fed state in a young fit human within thirty minutes, despite the teachings of the literature that exercise does not induce changes in plasma glycerol in young trained humans in the fed state during this time period (Coyle et al).

TABLE 1

| | | | Net change | |
|---|---|---|---|---|
| | | | in glycerol | |
| | Before | Glycerol | concentra- | |
| | or After | Concentra- | tion after | Percentage |
| Treatment | exercise | tion (μM) | exercise (μM) | change |
| A Trial 1 Placebo | Before | 40.0 | | |
| | After | 41.4 | 1.4 | 3.5 |
| A Trial 1 Treatment | Before | 26.2 | | |
| | After | 93.7 | 67.6 | 258.9 |
| A Trial 2 Placebo | Before | 34.5 | | |
| | After | 46.9 | 12.4 | 36.0 |
| A Trial 2 Treatment | Before | 31.7 | | |
| | After | 97.9 | 66.2 | 208.7 |
| A Trial 3 Treatment | Before | 19.3 | | |
| | After | 42.7 | 23.4 | 121.4 |
| B Trial 1 Placebo | Before | 64.8 | | |
| | After | 53.8 | −11.0 | −17.0 |
| B Trial 1 Treatment | Before | 35.8 | | |
| | After | 64.8 | 29.0 | 80.8 |
| B Trial 2 Placebo | Before | 44.1 | | |
| | After | 57.9 | 13.8 | 31.3 |
| B Trial 2 Treatment | Before | 30.3 | | |
| | After | 60.7 | 30.3 | 100.0 |
| B Trial 3 Treatment | Before | 22.1 | | |
| | After | 40.0 | 17.9 | 80.3 |
| C Trial 1 Placebo | Before | 48.6 | | |
| | After | 77.6 | 29.0 | 59.5 |
| C Trial 1 Treatment | Before | 42.7 | | |
| | After | 80.0 | 37.2 | 87.1 |
| C Trial 2 Placebo | Before | 37.1 | | |
| | After | 53.0 | 16.2 | 43.8 |
| C Trial 2 Treatment | Before | 52.1 | | |
| | After | 90.3 | 38.2 | 73.3 |
| C Trial 3 Treatment | Before | 27.6 | | |
| | After | 57.9 | 30.3 | 110.0 |

TABLE 2

| Concentration change (μM) | Placebo | Treatment |
|---|---|---|
| −11.0 | P | |
| 1.4 | P | |
| 12.4 | P | |
| 13.8 | P | |
| 16.2 | P | |
| 17.9 | | T |
| 23.4 | | T |
| 29.0 | P | |
| 29.0 | | T |
| 30.3 | | T |
| 30.3 | | T |
| 37.2 | | T |
| 38.2 | | T |
| 66.2 | | T |
| 67.6 | | T |

TABLE 3

| Percentage Change | Treatment | Placebo |
|---|---|---|
| −17.00 | | P |
| 3.45 | | P |
| 31.25 | | P |
| 36.00 | | P |
| 43.75 | | P |
| 59.52 | | P |
| 73.33 | T | |
| 80.77 | T | |
| 81.25 | T | |
| 87.10 | T | |
| 100.00 | T | |
| 110.00 | T | |
| 121.43 | T | |
| 208.70 | T | |
| 257.89 | T | |

TABLE 4
Average calories expended per kilogram from Precor calculator

| A | B | C |
|---|---|---|
| Mean 3.68 calories per | Mean 3.07 calories per | Mean 3.43 calories per |
| kg, median 3.66 cal/kg | kg, median 3.07 cal/kg | kilogram, median 3.45 |
| Standard error .12 | Standard error .016 | cal/kg |
| cal/kg | cal/kg | Standard error .081 |
| | | cal/kg |

Table 1 displays the Effect of TriAc on Exercise induced Lipolysis. Table 2 displays the Ranked changes in glycerol concentration after exercise ranked in order of magnitude. Table 3 displays Ranked percentage change from baseline in glycerol concentration after exercise ranked in order of magnitude. Table 4 shows the average calories expended per kilogram during exercise.

For the group, the concentration of glycerol increased an average of 12 micromolar, or 29% above baseline when this mild exercise occurred with placebo and increased an average of 37 micromolar, or 124% when exercise occurred with the TriAc treatment. Topical TriAc increased the exercise induced breakdown of fat as evidenced by plasma glycerol by over 300 percent. To put it another way, for the same amount of exercise, three times the amount of fat was broken down when TriAc creams were applied. Using percentage change in the concentration of glycerol, TriAc increases the effectiveness of exercise to burn fat by about 400%.

Although not shown in the Tables, when Triac was applied and no exercise occurred, two of the subjects had no response relative to baseline (13% (B) and −40% (C)) and the youngest subject (A) had about ⅓ of the response with exercise (80% with no exercise versus 250% with exercise) in one experiment.

The percentage changes are significantly different at a two fold p level of 0.01 using the Mann Whitney U statistic of 40 or 2.5, n=15. The percentage change from baseline is significantly different between treatment using the two tailed t-test for sample populations with either the same or unequal variances with a p value of less than 0.01. The change in glycerol concentration is significantly different using a one tailed t-test with unequal variances, at a p of 0.03. The change in glycerol concentration is different using a two tailed t-test with unequal variances, at a p of 0.058. Using either a one or two tailed t-test assuming equal variances in the populations, a p value of 0.01 or 0.005 is obtained. An F-test does not show any difference in variance in the population.

This example shows that application of topical TriAc before exercise significantly alters the human plasma glycerol level after exercise and thus topically applied TriAc and thyroid hormone like agonist analogs, can induce a significant degree of lipolysis in humans. The amount of thyroid hormone or thyroid hormone like agonist applied to the skin is below the amount of a single daily oral dose taken as a replacement or therapeutic dosage. The lipolytic effect can be maintained by using the topical preparation intermittently and before exercise.

All of the patents, publications and other references mentioned in this application are herein incorporated by reference in their entireties.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A TriAc topical preparation for treating burns or deep dermal ulcers in a human comprising less than 10 mg of TriAc per 100 ml of pharmaceutical excipient base.

2. The preparation of claim 1, wherein the preparation is applied intermittently.

3. The preparation of claim 2, wherein the preparation is applied less than once daily.

4. The preparation of claim 1, wherein the preparation delivers an amount of less than 500 ng per cm² of the burn.

5. The preparation of claim 4, wherein the preparation delivers an amount of less than 200 ng per cm² of the burn.

6. The preparation of claim 1, wherein the preparation is selected from the group consisting of: a) a semi solid cream; b) a liposomal preparation; c) a liposomal spray; d) a solid lipid nanoparticle; and e) a patch.

7. A unit dose package containing a single dose of the preparation of claim 1, wherein the single dose comprises less than 5 g of the preparation.

8. The preparation of claim 1, wherein the preparation comprises less than 1 mg of TriAc per 100 ml of pharmaceutical excipient base.

9. The preparation of claim 8, wherein the preparation is applied daily.

10. A pharmaceutical delivery system for treating a burn or a deep dermal ulcer in a human comprising TriAc delivered in an amount of less than 500 ng per cm² of the burn or the ulcer and a drug delivery device selected from the group consisting of a microneedle drug delivery device and a microchanneling RF drug delivery device.

11. The delivery system of claim 10, wherein an amount of less than 200 ng per cm² of the burn of TriAc is delivered.

12. (canceled)

13. (canceled)

14. (canceled)

15. (canceled)

16. (canceled)

17. (canceled)

18. (canceled)

19. A method of treating a burn or a deep dermal ulcer, comprising the steps of: a) providing a TriAc topical preparation comprising less than 10 mg of TriAc per 100 ml of pharmaceutical excipient base; and b) applying less than 500 ng of TriAc per cm² of the burn or the deep dermal ulcer to the burn or the deep dermal ulcer.

20. The method of claim 19, wherein step b) is repeated intermittently.

21. The method of claim 20, wherein step b) is repeated less than once a day.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A method of treating a human burn or a deep dermal ulcer, comprising the steps of: a) providing at least one agonist thyroid hormone compound or at least one agonist thyroid hormone like compound; and b) applying an amount of the at least one agonist thyroid hormone compound or the at least one agonist thyroid hormone like compound to the burn or the deep dermal ulcer, wherein the amount is sufficient to accelerate healing of the burn compared to an untreated burn or an untreated deep dermal ulcer, but the amount is less than an amount that would produce a change in at least one thyroid function test.

37. The method of claim 36, further comprising the step of repeating step b) intermittently.

38. The method of claim 36, wherein the thyroid hormone compound or the thyroid hormone like compound is applied less than once a day.

39. (canceled)

40. The preparation of claim 1, wherein the preparation reduces pain from a burn compared to a burn not treated with the preparation.

41. The method of claim 19, wherein the method reduces pain from a burn compared to a burn not treated with the method.

42. The method of claim 36, wherein the amount of the at least one agonist thyroid hormone compound or the at least one agonist thyroid hormone like compound is sufficient to decrease an amount of pain from the burn compared to an untreated burn.

43. A TriAc topical preparation for treating burns in a human comprising TriAc and a pharmaceutical excipient base, wherein the preparation reduces pain from a burn compared to a burn not treated with the preparation.
